(19) United States
(12) Patent Application Publication
(10) Pub. No.: US 2026/0144949 A1
(43) Pub. Date: May 28, 2026

(54) Inhalation System

(71) Applicant: General Biophysics LLC, Framingham, MA (US)

(72) Inventors: Ilya Ilin, Wayland, MA (US); George Hagopian, Boston, MA (US)

(21) Appl. No.: 19/362,824

(22) Filed: Oct. 20, 2025

Related U.S. Application Data
(60) Provisional application No. 63/709,149, filed on Oct. 18, 2024.

(51) Int. Cl. A61M 16/00; A61M 16/08; A61M 16/10; A61M 16/12; A61M 16/20

(52) U.S. Cl. CPC A61M 16/024; A61M 16/0003; A61M 16/0891; A61M 16/1015; A61M 16/104; A61M 16/12; A61M 16/208; A61M 2016/0033; A61M 2016/1025; A61M 2016/103; A61M 2202/0007; A61M 2202/0208; A61M 2202/0241; A61M 2202/0291; A61M 2205/3327; A61M 2205/3344; A61M 2205/505; A61M 2230/005; A61M 2230/40

(57) ABSTRACT

An automatic inhalation system which includes a gas supply system which includes a high pressure system and a medium pressure system, a breathing loop; wherein gas enters the breathing loop from the gas supply system; and wherein the breathing loop further comprises an inhale limb and an exhale limb each comprising at least one flow sensor, at least one CO₂ sensor, and at least one O₂ sensor. The system further includes a controller, wherein the gas supply system and breathing loop are in digital communication with the controller via an I2C bus and a set of analog inputs; and a touchscreen, wherein the touchscreen displays the diagnostic information on a UI accessed through a password-protected user management system; wherein the system is used to administer gas treatments to patients based on a preset protocol that may be started by either an operator or administrator and edited only by an administrator.

### CROSS REFERENCE TO RELATED APPLICATION

This application claims the benefit of and takes priority from U.S. Provisional Patent Application Ser. No. 63/709,149 filed on Oct. 18, 2024, the contents of which are herein incorporated by reference.

### BACKGROUND OF THE INVENTION

### Field of the Invention

The present disclosure relates generally to inhalation systems, devices and methods. More particularly, exemplary embodiments of the disclosure include an inhalation system and corresponding method that utilizes an automatic preset protocol and various safeguards to safely and effectively administer therapeutic xenon treatments.

### Description of the Related Art

The use of inhalation machines to deliver gases for analgesic purposes has a long and evolving history, shaped by the discovery and clinical application of agents like nitrous oxide (N₂O) and xenon. These machines are designed not for full surgical anesthesia, but for providing controlled, reversible pain relief and mild sedation-particularly during procedures that do not require loss of consciousness.

To safely and effectively deliver analgesic gases, inhalation machines were developed to allow administration of the gases via masks or mouthpieces. These machines include basic flow regulators, demand valves, and scavenging systems to control dosage and minimize exposure to staff.

First studied for its anesthetic potential in the 1950s, xenon is an inert noble gas that provides rapid induction and recovery, is non-toxic, and offers neuroprotective effects. However, its use has been limited due to high production costs and the need for specialized equipment to recycle the gas efficiently within the anesthesia circuit.

### SUMMARY OF THE INVENTION

The instant system and corresponding method, as illustrated herein, is clearly not anticipated, rendered obvious, or even present in any of the prior art mechanisms, either alone or in any combination thereof. Thus, the several embodiments of the instant system and method are illustrated herein.

A primary object of the present disclosure is to provide a system preferably for the safe administration of therapeutic xenon treatments using an automatic preset protocol wherein the protocol includes an optional denitrogenation phase, a xenon treatment phase using a preset subanesthetic concentration, and a dexenonation and a recovery phase.

Another object of the present disclosure is to provide a set of safeguards to ensure the safety of the patient receiving the xenon treatment including two oxygen sensors for inhale and exhale limbs, a CO₂ sensor that can measure on the level of parts per million (ppm), an automatic normally-opened safety valve that opens to the atmosphere, and an oxygen flush.

In one aspect of one preferred embodiment, an inhalation system is disclosed that includes a gas supply system and breathing loop coupled with a controller, wherein the gas supply system and breathing loop provide diagnostic information to the controller which displays the information on a touchscreen.

Furthermore, in one embodiment, the gas supply system, breathing loop, and controller are contained within a housing defined by a front, a back, a left side, a right side and a top, or upper portion comprising a gradient or slant disposed for securely receiving the touchscreen. In one embodiment, the top also comprises an oxygen flush actuation button, within the expiratory flow path which allows the user to flood the system with oxygen to prevent hypoxia.

Moreover, in one embodiment, the gas supply system includes a high-pressure system and a medium pressure system, wherein the high-pressure system feeds gas from oxygen gas cylinder supplies and a xenon gas cylinder supply into the medium pressure system via two separate subsystems: an oxygen high pressure system and a xenon high pressure system. The high-pressure system preferably steps down the pressure of the gas from the oxygen gas cylinder supplies and the xenon gas cylinder supply before providing it to the medium pressure system using an oxygen pressure regulator.

In one embodiment, the flow of gas into the medium pressure system is monitored by a flow rate sensor that sends information to the controller, which in turn displays the information on the touchscreen. The medium pressure system further comprises separate gas supply lines that provide the gas to the breathing loop, wherein the oxygen gas from the oxygen high pressure system and the xenon gas from the xenon high pressure system are not mixed in the medium pressure system.

In one embodiment, the breathing loop comprises a set of limbs, specifically an inhale and exhale limb wherein each limb comprises sensors to monitor the gas being delivered to the patient and that being exhaled by the patient, including, but not limited to flow sensors, CO₂ sensors, O₂ sensors, pressure sensors and Xe sensors. The breathing loop further Comprises an automatic normally-open safety valve that can allow fresh air to enter the breathing loop in the case of a power or gas supply malfunction.

In one preferred embodiment, two O₂ sensors will be utilized. By employing these two sensors, the system is able to calculate the rate of O2 consumption of the patient and use this information to adjust the background O2 flow accordingly. This feature allows the system to minimize the amount of O2 used and as a result minimize the amount of xenon needed to be added to the circuit in order to maintain a stable concentration.

Additionally, the employment of O₂ sensors serves an additional safety feature as, in the case where the patient has a higher-than-normal rate of oxygen consumption, the system can increase the background flow rate to match the needs of the individual patient.

In one embodiment, the breathing loop comprises an optional portable recovery system that can capture and concentrate xenon gas to a level higher than the administered concentration during the patient treatment procedure.

Another object of the present disclosure is to provide a password-protected user management system, wherein operators cannot alter the settings of the system. The password protection combined with the previously mentioned safeguards make it possible to administer a treatment without the presence of an anesthesiologist. Furthermore, the password-protected user management system can allow certain users to alter the settings based on their credentials, if so required.

There has thus been outlined, rather broadly, the more important features of an inhalation system and associated method, in order that the detailed description thereof that follows may be better understood, and in order that the present contribution to the art may be better appreciated. There are additional features of the system that will be described hereinafter and which will form the subject matter of the claims appended hereto.

In this respect, before explaining at least one embodiment of the system in detail, it is to be understood that the system is not limited in its application to the details of construction and to the arrangements of the components set forth in the following description, and/or illustrated in the drawings. The system is capable of other embodiments and of being practiced and carried out in various ways. Also, it is to be understood that the phraseology and terminology employed herein are for the purpose of description and should not be regarded as limiting.

These together with other objects of the system, along with the various features of novelty, which characterize the system, are pointed out with particularity in the claims annexed to and forming a part of this disclosure. For a better understanding of the system, its operating advantages and the specific objects attained by its uses, reference should be made to the accompanying drawings and descriptive matter in which there are illustrated preferred embodiments of the system.

The foregoing has outlined the more pertinent and important features of the present system in order that the detailed description of the system that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

### BRIEF DESCRIPTION OF THE DRAWINGS

This invention may be more completely understood in consideration of the following detailed description of various embodiments of the invention in connection with the accompanying drawings, in which:

FIGS. 1A-1C illustrate a front, left perspective, and right perspective view of the inhalation system, respectively.

FIG. 2 illustrates a box diagram of the gas supply system.

FIG. 3 illustrates a box diagram of the oxygen high pressure system.

FIG. 4 illustrates a box diagram of the xenon high pressure system.

FIG. 5 illustrates a box diagram of the medium pressure system.

FIGS. 6A-6B illustrate an enlarged view of the gas supply system.

FIGS. 7A-7B illustrate an enlarged view of the cylinder holding space.

FIGS. 8A-8B illustrate a box diagram of the medium pressure system side by side with a perspective view of the medium pressure system.

FIG. 9 illustrates a box diagram of the breathing loop.

FIGS. 10A-10B illustrate an enlarged view of the breathing loop.

FIGS. 11A-11B illustrate a box diagram of the breathing loop side by side with a perspective view of the breathing loop.

FIG. 12 illustrates a wiring diagram of the inhalation system.

FIGS. 13A-13B illustrate an enlarged view of the I2C PCB.

FIGS. 14A-14B illustrate an enlarged view of the analog interface PCB.

FIG. 15 illustrates a top perspective view of the housing of the inhalation system.

FIGS. 16A-16D illustrate a password-protected management system displayed on the touchscreen.

FIGS. 17A-17E illustrate the user interface of the inhalation system.

FIG. 18 illustrates a schematic of the I2C PCB.

FIG. 19 illustrates a schematic of the analog interface PCB.

### DETAILED DESCRIPTION OF THE SEVERAL EMBODIMENTS

The detailed description set forth below in connection with the appended drawings is intended as a description of several embodiments of the apparatus and does not represent the only forms in which the present apparatus may be constructed and/or utilized. The description sets forth the functions and the sequence of steps for constructing and operating the apparatus in connection with the illustrated embodiments. However, it is to be understood that the same or equivalent functions and sequences may be accomplished by different embodiments that are also intended to be encompassed within the spirit and scope of the invention.

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

Reference will now be made to non-limiting embodiments, examples of which are illustrated in the Figures.

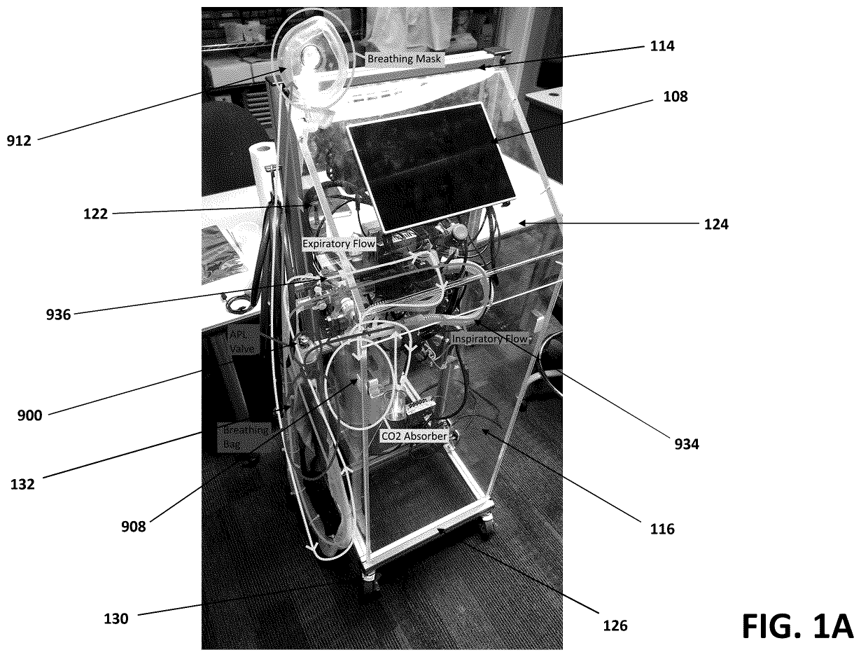

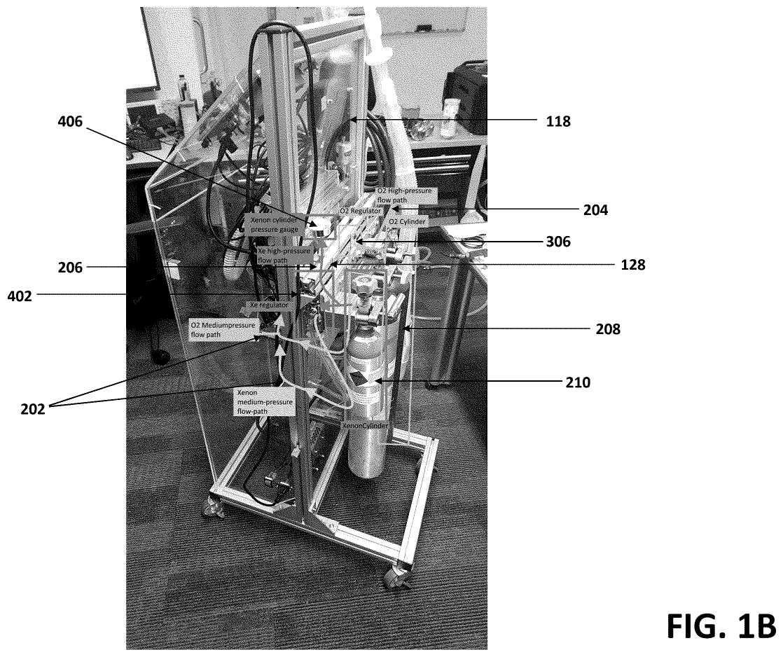

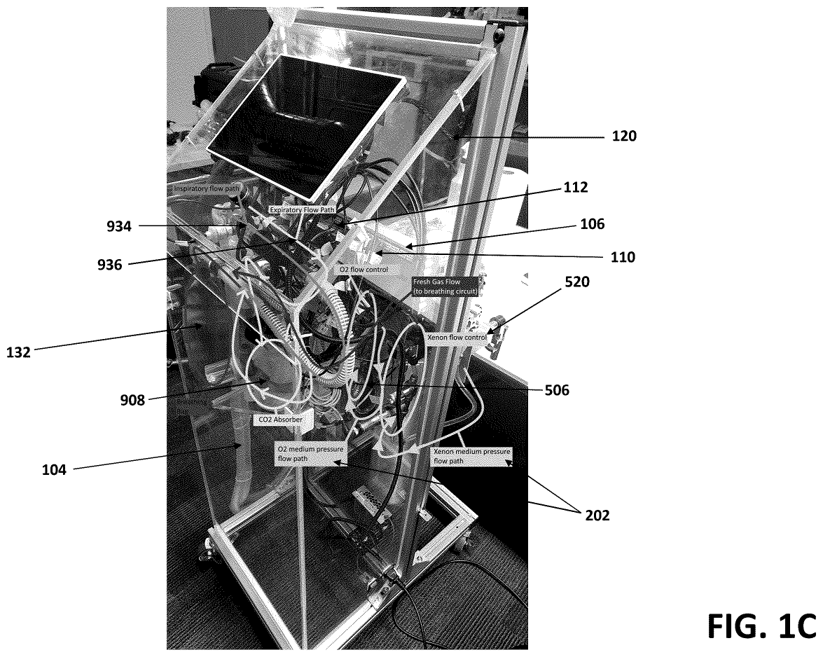

FIGS. 1A-1C illustrate various perspectives of an automatic inhalation system 100, wherein the automatic inhalation system 100 preferably comprises a gas supply system 102, a breathing loop 104, a controller 106, and a touchscreen 108. The automatic inhalation system 100 may be powered through conventional power means through a standard electrical outlet or similar means. Preferably, the gas supply system 102 and the breathing loop 104 are in digital communication with the controller 106 via an I2C bus 110 and a set of analog inputs 112.

In some embodiments, the automatic inhalation system 100 is comprised within a housing 114. In some embodiments, portions of the automatic inhalation system 100, specifically, portions of the breathing loop 104, may reside outside of the housing 114. In one preferred embodiment, the housing 114 is preferably between 4.5 and 5 feet tall and defined by a front 116, back 118, left side 120, right side 122, and slanted top 124. The housing 114 further comprises a base 126, and a cylinder holding space 128. The base 126 further comprises a set of wheels 130 and should be large enough to provide stability. The cylinder holding space 128 resides on the outer side of the back 118 and is preferably large enough to hold up to three gas cylinders. Moreover, the touchscreen 108 is preferably mounted on the outside of the slanted top 124 of the housing 114 for easy access.

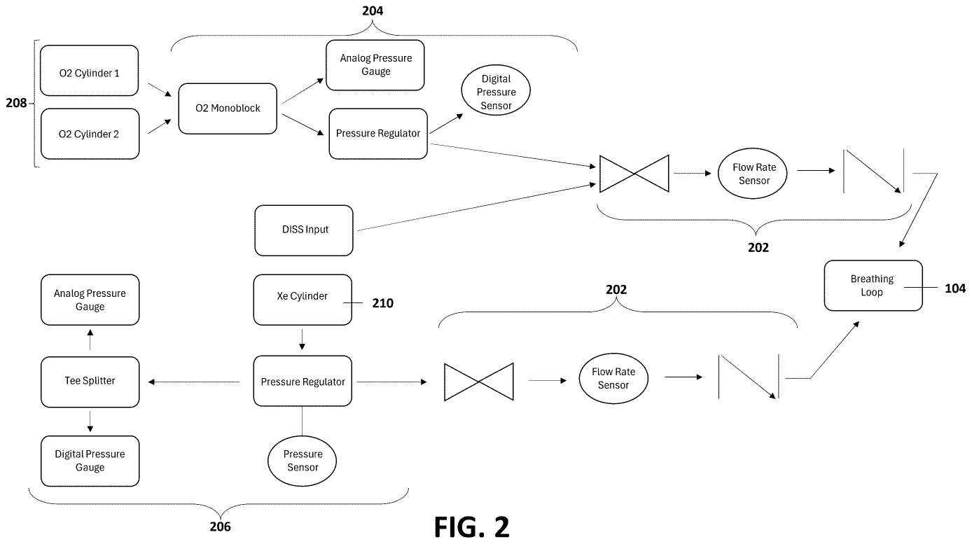

FIG. 2 illustrates a box diagram of the gas supply system 102. The gas supply system 102 comprises the high-pressure system 200, and the medium pressure system 202. The high-pressure system 202 further comprises the oxygen high pressure system 204 and the xenon high pressure system 206. The gas is routed to move from the oxygen gas cylinder supplies 208 and the xenon gas cylinder supply 210 through the gas supply system 102 in order to reach the breathing loop 104.

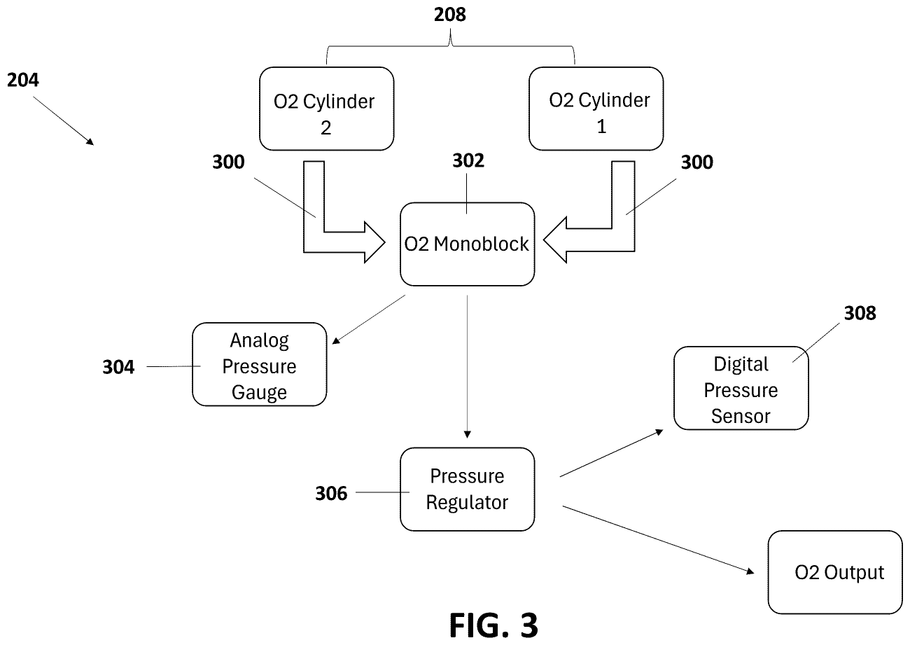

FIG. 3 illustrates a box diagram of the oxygen high pressure system 204. The oxygen high pressure system 204 preferably comprises the oxygen gas cylinder supplies 208 connected through a gas yoke 300 to a stainless-steel oxygen monoblock 302. In one preferred embodiment, the gas yoke 300 is a CGA 2.5 gas yoke. The oxygen monoblock 302 further connects to an analog pressure gauge 304 and an oxygen pressure regulator 306 via brass NPT fittings. The analog pressure gauge 304 visually displays the amount of gas left in the oxygen gas cylinder supplies 208, while the oxygen pressure regulator 306 steps the pressure of the incoming gas down. In one preferred embodiment, the gas fed into the oxygen pressure regulator 306 from oxygen gas cylinder supplies 208 may operate at a pressure of 3000 psi.

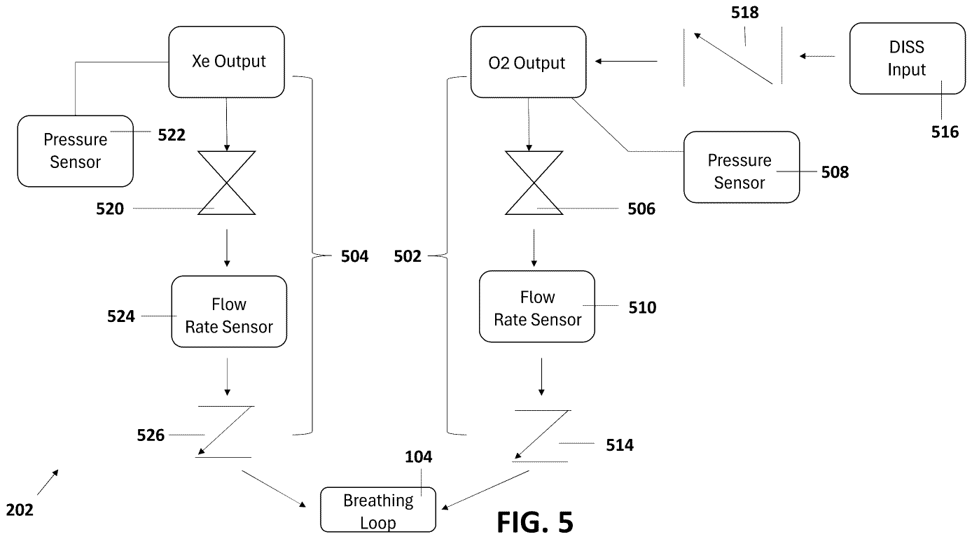

In this embodiment, the oxygen pressure regulator 306 drops the pressure of the gas to 50-60 psi. The gas, now at a pressure of 50-60 psi, can be fed into the medium pressure system 202 (shown in FIG. 5). The oxygen pressure regulator 306 additionally has a secondary port to connect to a digital pressure sensor 308. The digital pressure sensor 308 measures the pressure of the set of oxygen gas cylinder supplies 208 and feeds that information to the controller 106.

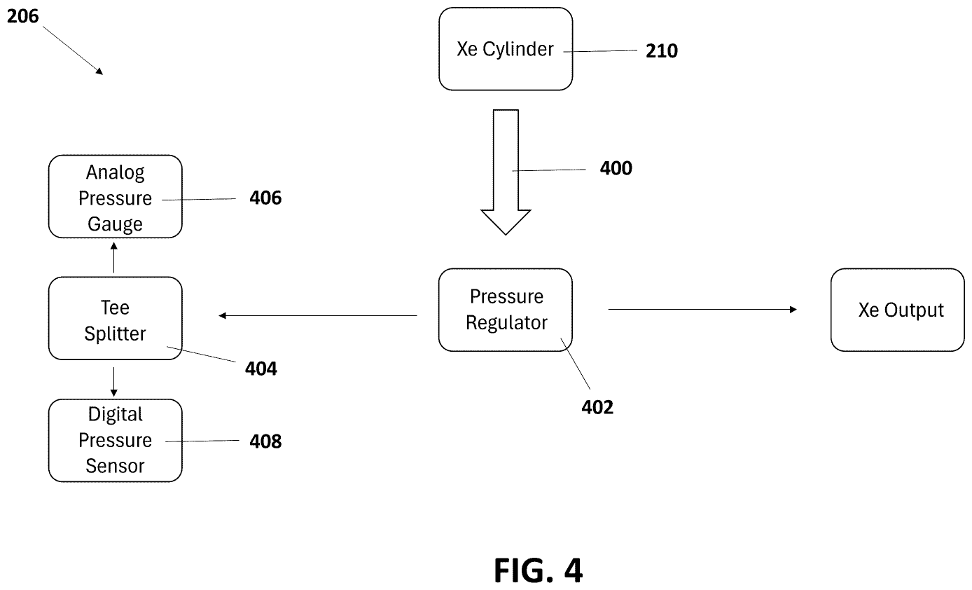

FIG. 4 illustrates a box diagram of the xenon high pressure system 206. The xenon high pressure system 206 preferably comprises the xenon gas cylinder supply 210 connected through a gas yoke 400 to a xenon pressure regulator 402. In one preferred embodiment, the gas yoke 400 may comprise a CGA 971 gas yoke. The xenon pressure regulator 402 is further connected to an alternate port that is split with a tee fitting 404 into an NPT port of an analog pressure gauge 406 and an NPT port of a digital pressure sensor 408. The analog pressure gauge 406 visually displays the gas level or amount of gas left in the xenon gas cylinder supply 210, while the digital pressure sensor 408 measures the xenon gas cylinder supply 210 pressure. The digital pressure sensor 408 then feeds this information to the controller 106. In this embodiment, the gas fed into the xenon pressure regulator 402 from the xenon gas cylinder supply 210 has a pressure of 600-750 psi. The xenon pressure regulator 402 steps down the pressure of the gas to 50-60 psi.

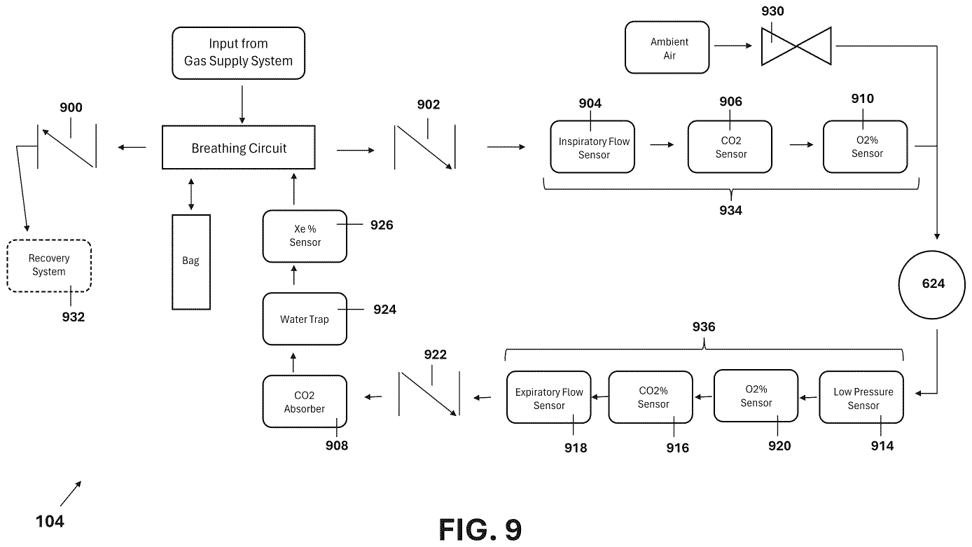

FIG. 5 illustrates a box diagram of the medium pressure system 202. The medium pressure system 202 comprises a set of gas supply lines 500 that are responsible for dispensing gas into a patient breathing loop 104 (shown in FIG. 9). The set of gas supply lines 500 preferably comprises an oxygen supply line 502 and a xenon supply line 504. Gas output from the oxygen pressure regulator 306 enters the medium pressure system 202 through a proportional solenoid valve 506. In this embodiment, the proportional solenoid valve 506 works only at a pressure of 50-60 psi. To verify that the gas at the proportional solenoid valve 506 is at the correct pressure, an oxygen pressure sensor 508 sends an electrical signal to the controller 106 proportional to the amount of pressure at the proportional solenoid valve 506 input.

Moreover, the proportional solenoid valve 506 is preferably controlled by an electrical signal originating from the controller 106. The amount the proportional solenoid valve 506 opens is proportional to the amount of power Supplied to it by the controller 106. The amount of power supplied to the proportional solenoid valve 506 varies how much the proportional solenoid valve 506 opens, and therefore, how much gas flows through. In one preferred embodiment, the proportional solenoid valve 506 is calibrated to allow gas to flow through at a rate of up to 15 liters per minute.

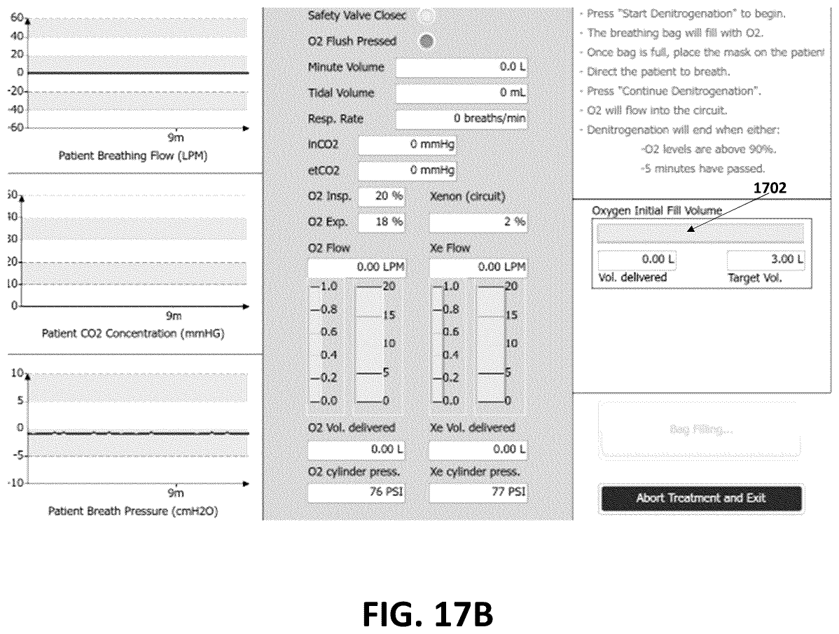

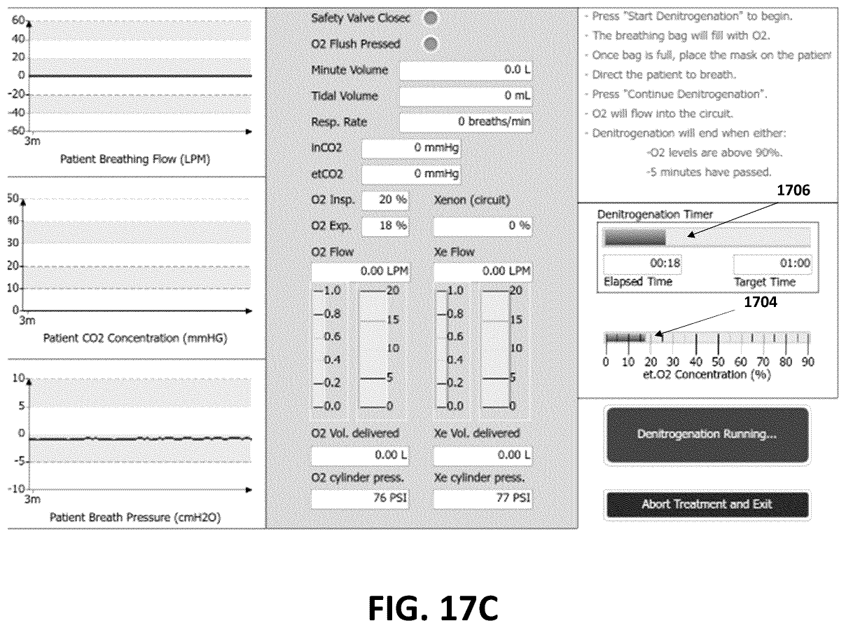

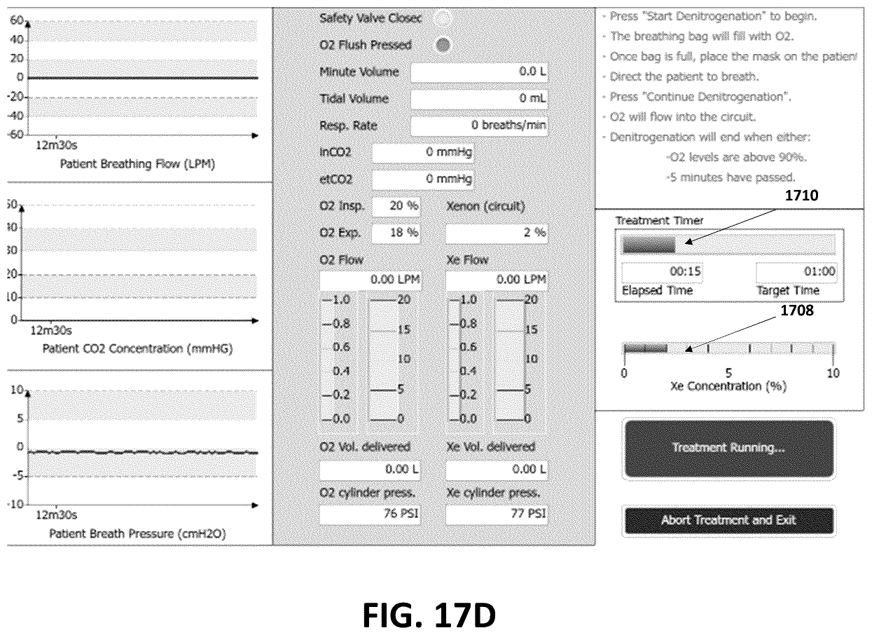

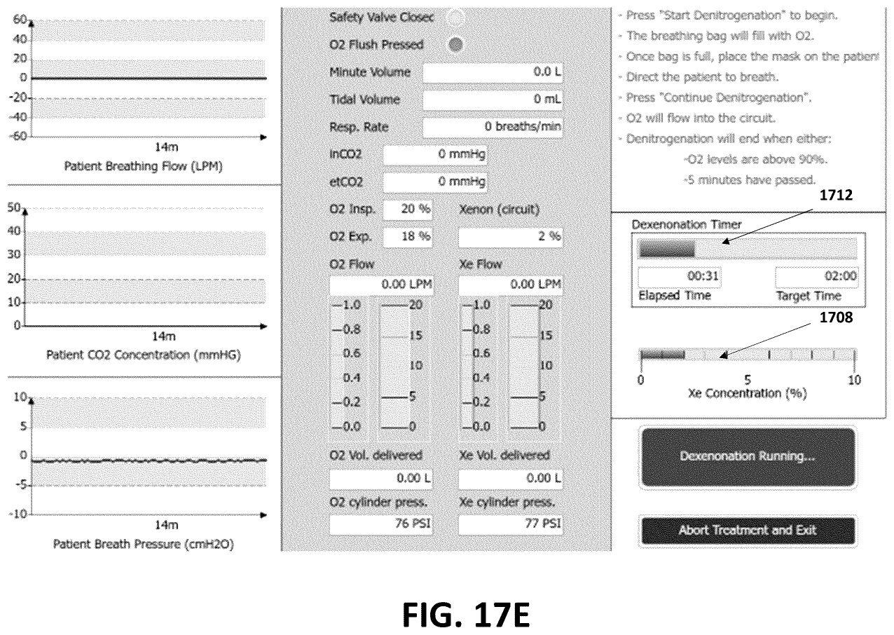

The gas flow rate is verified by a flow sensor 510 that is placed after the proportional solenoid valve 506 in the oxygen supply line 502. The flow sensor 510 continuously sends data to the controller 106. A user can read the data sent to the controller 106 from the flow sensor 510 in a user interface 512 (shown in FIGS. 17A-17E) in order to verify the amount of gas being dispensed through the proportional solenoid valve 506 and adjust the amount being dispensed by adjusting the power supplied to the proportional solenoid valve 506 by the controller. This feedback loop ensures accurate and consistent gas delivery.

After going through the flow sensor 510, the gas in the oxygen supply line 502 is fed through a check valve 514 into the breathing loop 104. The check valve 514 prevents back flow through the oxygen supply line 502.

In one preferred embodiment, the inhalation system 100 also comprises a DISS input port 516 for connecting a hospital oxygen supply. In this embodiment, the DISS input port 516 may provide oxygen to the medium pressure system 202 through the proportional solenoid valve 506. A check valve 518 is present between the DISS input port 516 and the proportional solenoid valve 506 to prevent gas loss from the inhalation system 100 if the DISS input port 516 is left open.

Gas output from the xenon pressure regulator 402 enters the medium pressure system 202 through a proportional solenoid valve 520. In this embodiment, the proportional solenoid valve 520 works only at a pressure of 50-60 psi. To verify that the gas at the proportional solenoid valve 520 is at the correct pressure, a xenon pressure sensor 522 sends an electrical signal to the controller 106 proportional to the amount of pressure at the proportional solenoid valve 520 input.

Moreover, the proportional solenoid valve 520 is preferably controlled by an electrical signal originating from the controller 106. The amount the proportional solenoid valve 520 opens is proportional to the amount of power supplied to it by the controller 106. The amount of power supplied to the proportional solenoid valve 520 varies how much the proportional solenoid valve 520 opens, and therefore, how much gas flows through. In one preferred embodiment, the proportional solenoid valve 520 is calibrated to allow gas to flow through at a rate of up to 15 liters per minute.

The gas flow rate is verified by a flow sensor 524 that is placed after the proportional solenoid valve 520 in the xenon supply line 504. The flow sensor 524 continuously sends data to the controller 106. A user can read the data sent to the controller 106 from the flow sensor 524 in the user interface 512 in order to verify the amount of gas being dispensed through the proportional solenoid valve 520 and adjust the amount being dispensed by adjusting the power supplied to the proportional solenoid valve 520 by the controller 106. This feedback loop ensures accurate and consistent gas delivery.

After going through the flow sensor 524, the gas in the xenon supply line 504 is fed through a check valve 526 into the breathing loop 104. The check valve 526 prevents back flow through the xenon gas line 504.

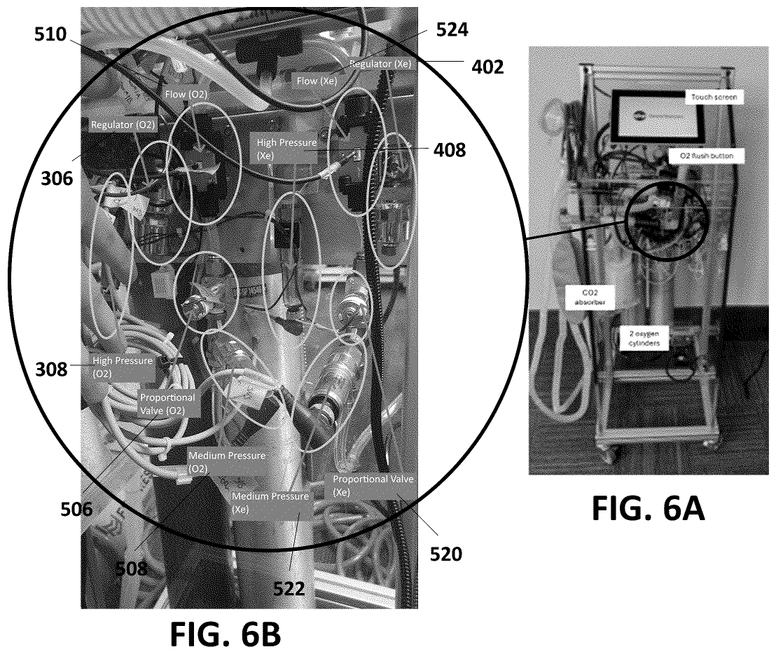

FIGS. 6A-6B illustrate an enlarged view of the gas supply system 102. The gas supply system 102 resides within the housing 114 with its various sensors and valves attached to the inside of the back 118 of the housing 114.

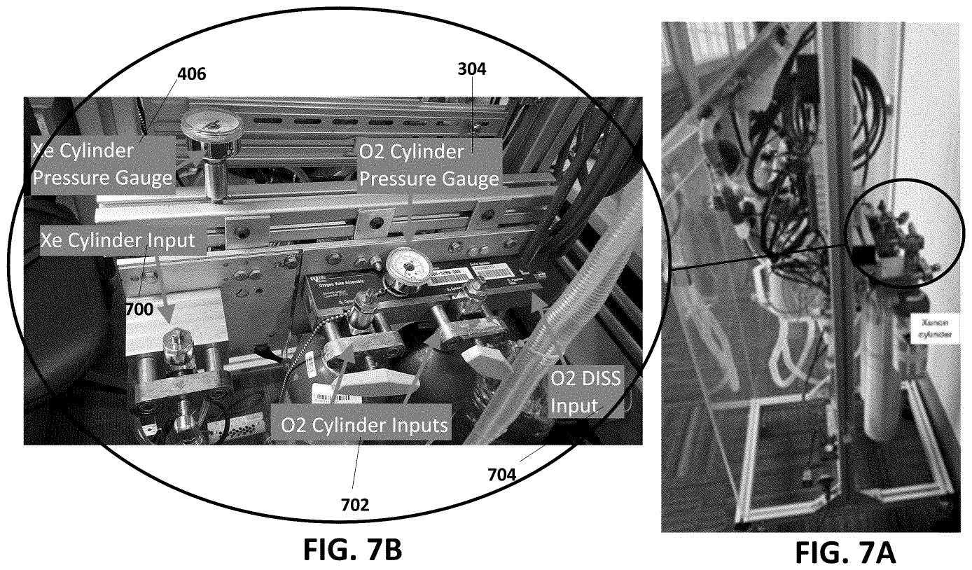

FIGS. 7A-B illustrate an enlarged view of the cylinder holding space 128. The cylinder holding space 128 resides on the outside of the back 118 of the housing 114. Specifically, the cylinder holding space 128 comprises the analog pressure gauges 304, 406 an Xe cylinder input 700, O₂ cylinder inputs 702, and an O₂ DISS input 704. The oxygen gas cylinder supplies 208 attach to the inhalation system at the O₂ cylinder inputs 702 and the xenon gas cylinder supply 210 attach to the inhalation system at the Xe cylinder input 700.

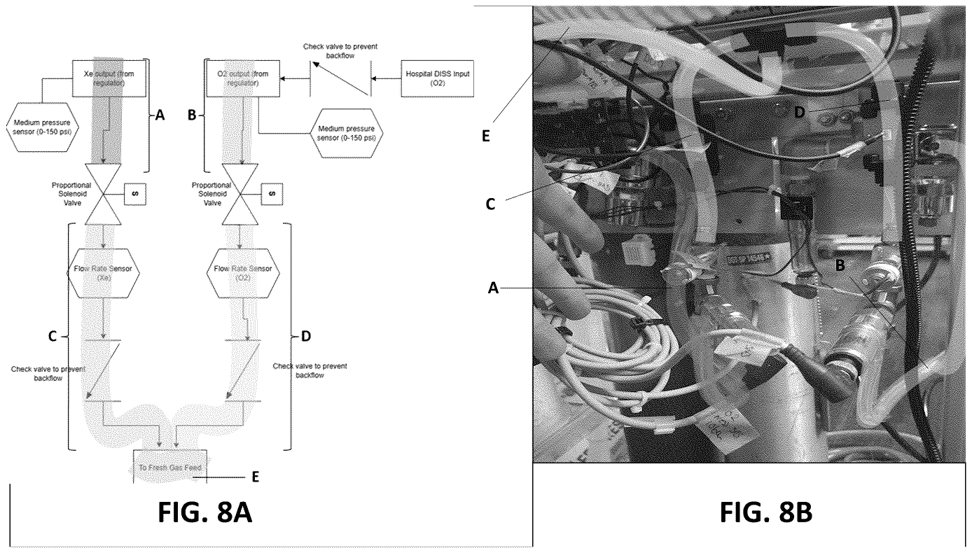

FIGS. 8A-8B illustrate a box diagram of the medium pressure system 202 side by side with a perspective view of the medium pressure system 202. The main components of the medium pressure system 202 are labeled alphabetically in order to show where the component can be found in one embodiment of the inhalation system 100.

FIG. 9 illustrates a box diagram of the breathing loop 104. The breathing loop 104 operates via spontaneous respiration, meaning that the patient controls their own intake of gas. The breathing loop 104 comprises an adjustable pressure limiting (APL) valve 900 that sets the maximum pressure limit on the breathing loop 104. Preferably, the pressure can be adjusted between 20-850 cm H₂O. The APL valve 900 can be opened if a patient breathing bag 132 is full. The gas is delivered to the breathing loop 104 from the oxygen supply line 502 and the xenon supply line 504. After entering the breathing loop 104, the gas goes through an inspiratory check valve 902, which serves to control the direction of gas flow in the breathing loop 104, preventing backflow. The gas then reaches an inhale limb 934 comprising an inspiratory flow sensor 904. The inspiratory flow sensor 904 monitors the inhaled flow rate of the patient. This information is fed to the controller 106 for the user to monitor on the touchscreen 108 as it is important diagnostic information and allows the user to verify that the patient is breathing normally.

As an additional safety measure, the inhale limb 934 further comprises a CO₂ sensor 906 that can measure on the level of parts per million (ppm). The CO₂ sensor 906 should read 0 ppm. Any elevated value means that a CO₂ absorber 908, found later in the breathing loop 104, is not working. The CO₂ sensor 906 sends this data to the controller 106 to be displayed on the touchscreen 108. The inhale limb 934 also preferably comprises an inspiratory O₂ percentage sensor 910. The inspiratory O₂ percentage sensor 910 monitors the amount of oxygen in the inspiratory gas. The gas then reaches the patient via a mask 912.

Once the gas is exhaled by the patient, it continues through the breathing loop 104 into an exhale limb 936. Much like the inhale limb 934, the exhale limb 936 also comprises a set of sensors to monitor the treatment. One of these sensors is a low-pressure sensor 914. In one preferred embodiment, the low-pressure sensor 914 measures 1 to −1 psi, −70 to 70 cm H₂O. Typically, the patient should have a breathing pressure of about ±4 cm H₂O.

The exhale limb 936 further comprises a CO₂ percentage sensor 916 which monitors the percentage of CO₂ in the patient's exhale. Typically, the patient's exhale will have a CO₂ percentage of about 4-5%. An expiratory flow sensor 918 is also comprised by the exhale limb 936. The expiratory flow sensor 918 monitors the exhaled flow rate of the patient. Similarly, there is an expiratory O₂ percentage sensor 920 within the exhale limb 936. The expiratory O₂ percentage sensor 920 measures the percentage of the O2 in the exhaled gas.

The data collected by the low-pressure sensor 914, the CO₂ percentage sensor 916, the expiratory flow sensor 918, and the expiratory O₂ percentage sensor 920 is fed to the controller 106 to be displayed on the touchscreen 108 for the user to monitor. After passing through the exhale limb 936, the exhaled gas goes through an expiratory check valve 922 which serves to control the direction of gas flow in the breathing loop 104, preventing backflow.

Moreover, when the patient exhales, their exhaled breath contains a high concentration of CO₂, which would be dangerous for them to rebreathe. Therefore, to prevent the patient from breathing in their own exhaled CO₂, after going through the expiratory check valve 922, the exhaled air is scrubbed of CO₂ via the CO₂ absorber 908. Additionally, a water trap 924 is present in the breathing loop 104 following the CO₂ absorber 908 to collect moisture that may be present in the breathing loop 104. In one preferred embodiment, the breathing loop 104 also comprises a xenon percentage sensor 926 after the water trap 924. The xenon percentage sensor 926 measures the amount of the xenon present in the exhaled gas. The data collected by the xenon percentage sensor 926 is fed to the controller 106 to be displayed on the touchscreen 108 for the user to monitor.

In one preferred embodiment, the breathing loop 104 comprises an oxygen flush 928. The oxygen flush 928 allows the user to flood the breathing loop 104 with a large flow of oxygen to prevent potential hypoxia. The breathing loop 104 also preferably comprises an automatic normally-open safety valve 930. The automatic normally-open safety valve 930 allows the treatment to be interrupted by providing atmospheric air to the patient. The atmospheric air can enter the breathing loop 104 through the automatic normally-open safety valve 930. This may be necessary in the case of a power outage or if the oxygen gas cylinder supplies 208 or the xenon gas cylinder supply 210 runs out of gas. This is unlike traditional anesthetic machines where the patient must stay under anesthesia in the case of any malfunction with the gas or power supply.

In some embodiments, the APL valve 900 vents to a recovery system 932. The recovery system 932 is designed to allow the re-use of xenon gas for multiple patients. In one embodiment, the recovery system 932 acts a buffer vessel. In such an embodiment, all of the gas exhaled by the patient is compressed and collected. The xenon concentration of the collected gas is measured by the recovery system 932 and collection ends when the concentration of the xenon gas drops to 0% or some other threshold value, such as 5%. Once the collection ends, the exhaled gas is vented to the atmosphere. The collected gas, with a concentration of 10%, is preferably then compressed into a holding cylinder, cleaned by a HEPA filter, and directed to the same or different patient in the next treatment procedure. In this embodiment, only half of the fresh xenon gas is needed to achieve the required concentration of 20% in the subsequent treatments as in the first.

In another embodiment, the recovery system 932 may comprise a recovery unit with a xenon selective absorbent or membrane. The selective absorbent absorbs the xenon gas from the exhaled gas, extracts is, cleans it using a HEPA filter, and directs it to the breathing loop 104 for the next treatment. In this embodiment, the recovery system 932 may comprise a buffer vessel to hold the exhaled gas or extract the exhaled gas directly from the breathing loop 104 on demand.

In yet another embodiment, the recovery system 932 comprises an absorbent cartridge that collects all exhaled gas following a treatment. The absorbent cartridge can then be shipped to a central facility for xenon extraction.

In some embodiments, the breathing loop 104 may also comprise a set of pneumatically operated pilot valves (not pictured). The set of pneumatically operated pilot valves would allow the user to switch the patient out of the breathing loop 104 to prevent gas delivery to the patient in the case of a hypoxic mixture. The set of pneumatically operated pilot valves could also allow a user to divert the patient's exhale into a recovery system 932. Normally, in this embodiment, the set of pneumatically operated pilot valves would be open to the atmosphere and would only switch the patient into the breathing loop 104 when the safety conditions were met.

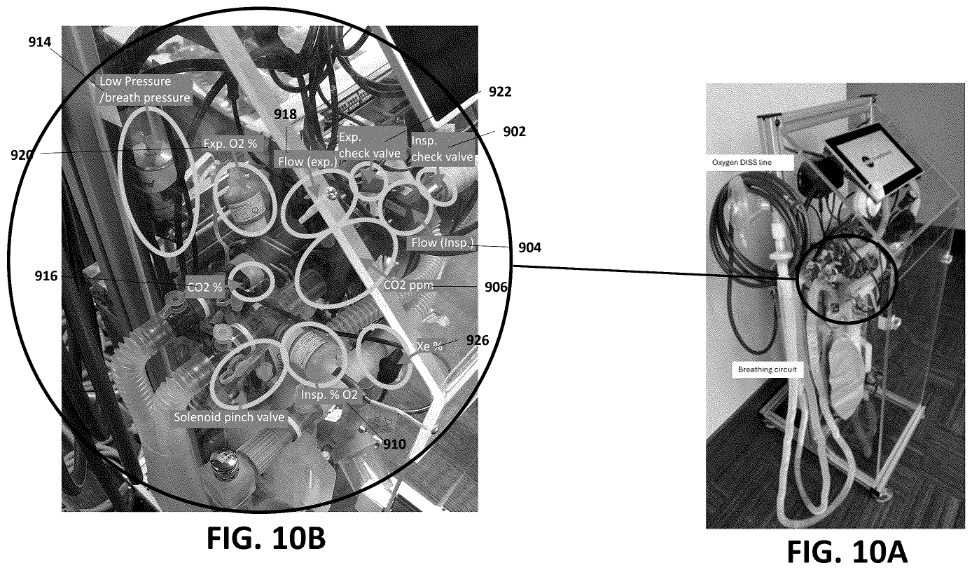

FIGS. 10A-10B illustrates an enlarged view of the breathing loop 104. In one preferred embodiment, the majority of the breathing loop 104 resides within the housing 114 with the patient breathing bag 132 on the outside of the housing 114. The patient breathing bag 132 and the oxygen flush 928 are preferably on the outside of the housing 114 to allow for easy access.

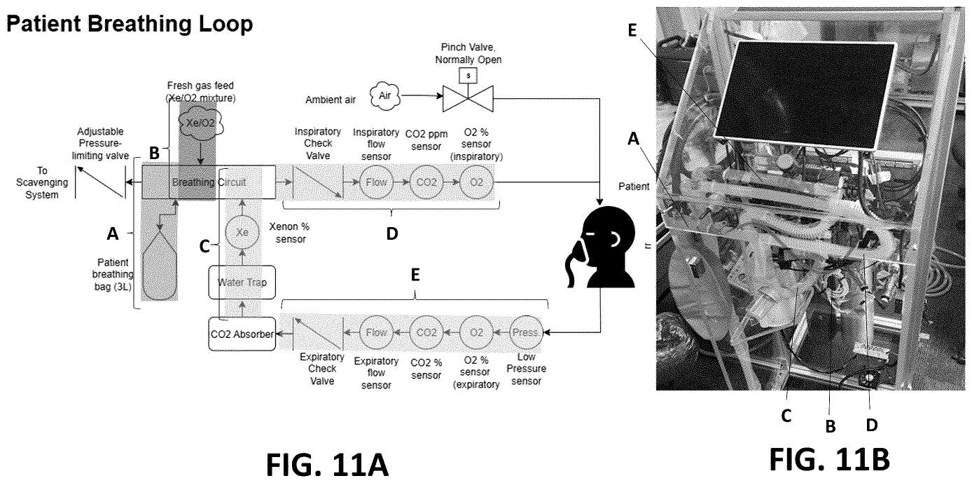

FIGS. 11A-11B illustrate a box diagram of the breathing loop 104 side by side with a perspective view of the breathing loop 104. The main components of the breathing loop 104 are labeled alphabetically in order to show where the component can be found in one embodiment of the inhalation system 100.

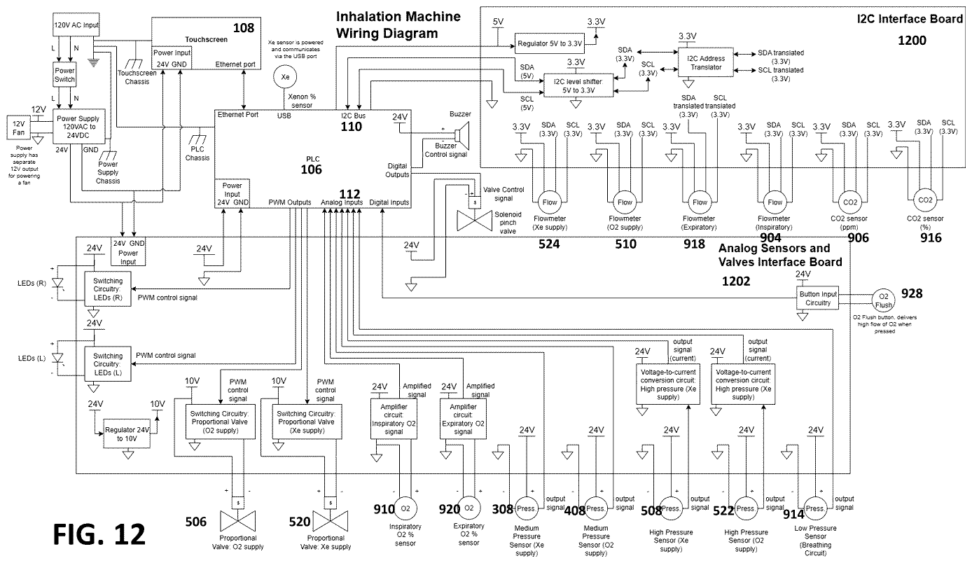

FIG. 12 illustrates a wiring diagram of the inhalation system 100. In one preferred embodiment, the inhalation system 100 comprises an I2C PCB 1200 and an analog interface PCB 1202. In this embodiment, the controller 102 is in digital communication with the I2C PCB 1200 via the I2C bus 110. The I2C PCB 1200 shares information with the controller 106 from all digital sensors including the flow sensors 508, 522 of the medium pressure system 202, the inspiratory flow sensor 904 of the breathing loop 104, the expiratory flow sensor 918 of the breathing loop 104, the CO₂ sensor 906 of the breathing loop 104, and the CO₂ percentage sensor 916 of the breathing loop 104.

Similarly, the analog interface PCB 1202 shares information with the controller 106 via the set of analog inputs 112. The information is from all analog sensors including the inspiratory O₂ percentage sensor 910, the expiratory O₂ percentage sensor 920, the xenon pressure sensor 520 of the medium pressure system 202, the oxygen pressure sensor 508 of the medium pressure system 202, the digital pressure sensor 308 of the oxygen high pressure system 204, the digital pressure sensor 404 of the xenon high pressure system 206, and the low pressure sensor 914 of the breathing loop 104. The controller 106 communicates with the analog interface PCB 1202 in order to read the raw voltage/current values from the analog sensors and converts these values to physical units.

After receiving the data from the I2C PCB 1200 and the analog interface PCB 1202, the controller 106 displays this information on the touchscreen 108 in the form of graphs and charts. Additionally, the controller 106 scans the touchscreen 108 for user input and performs the appropriate action if detected. In one preferred embodiment, user input is given by touching one touchscreen 108.

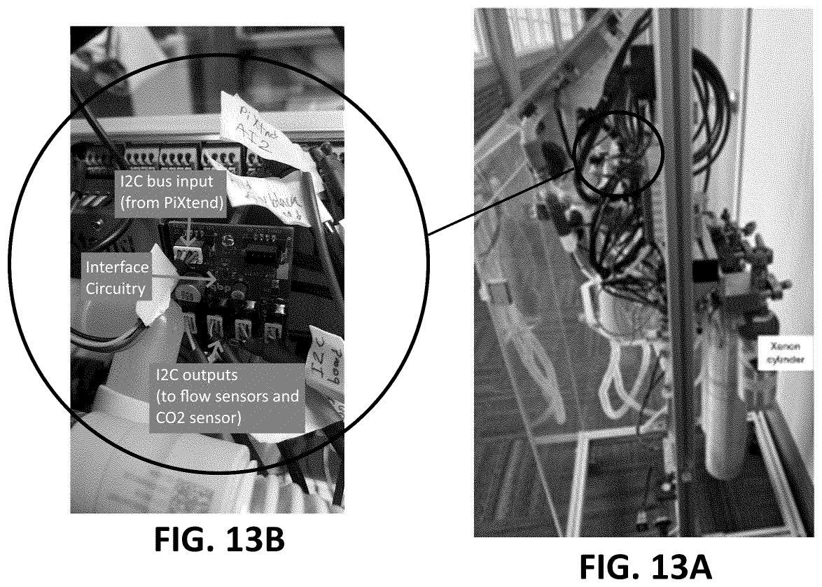

FIG. 13 illustrates an enlarged view of the I2C PCB 1200. In one preferred embodiment, the I2C PCB 1200 resides within the housing 114, attached to the inside of the back 118.

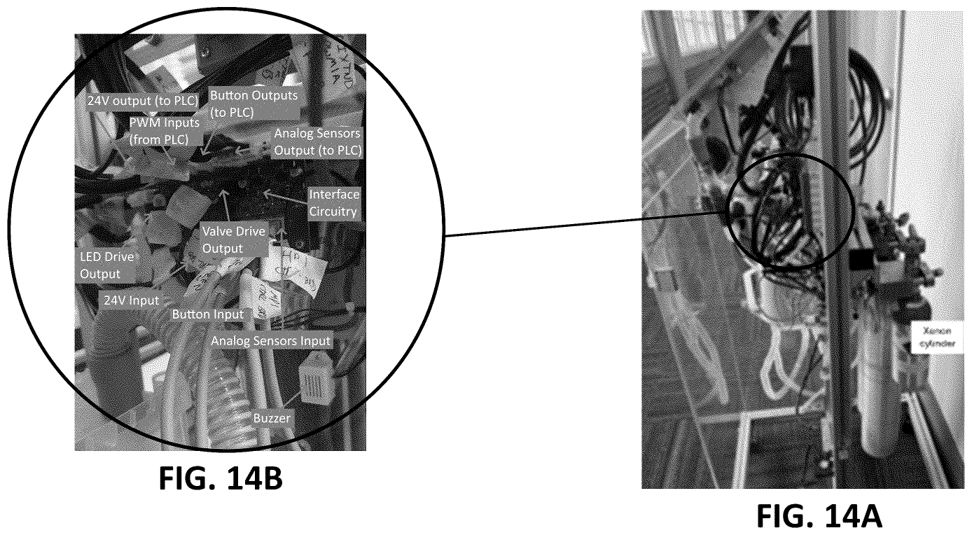

FIG. 14 illustrates an enlarged view of the analog interface PCB 1202. In one preferred embodiment, the analog interface PCB 1202 resides within the housing 114, attached to the inside of the back 118, and below the I2C PCB 1200.

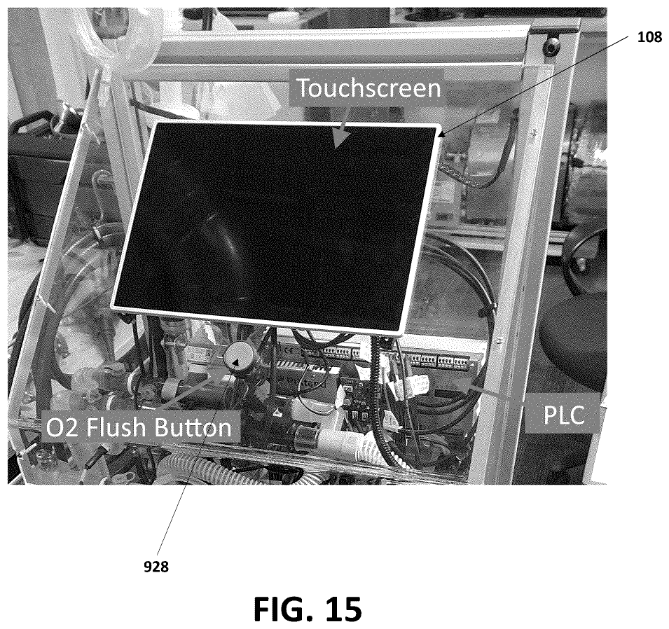

FIG. 15 illustrates a perspective view of the slanted top 124 of the housing 114 of the inhalation system 100. Attached to the slanted top 124 is the touchscreen 108. The touchscreen 108 portrays information provided by the controller 106 in the form of charts and graphs. Additionally, below the touchscreen 108 on the slanted top 124 is the oxygen flush 928.

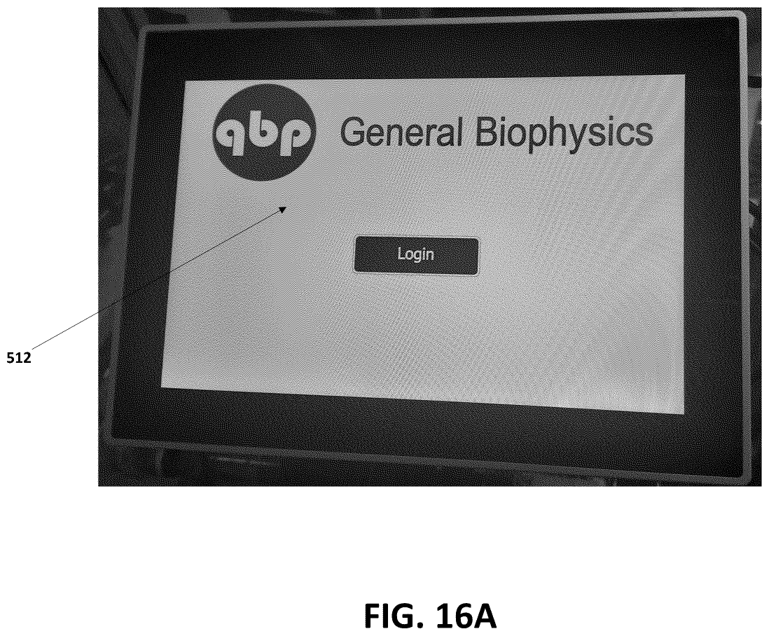

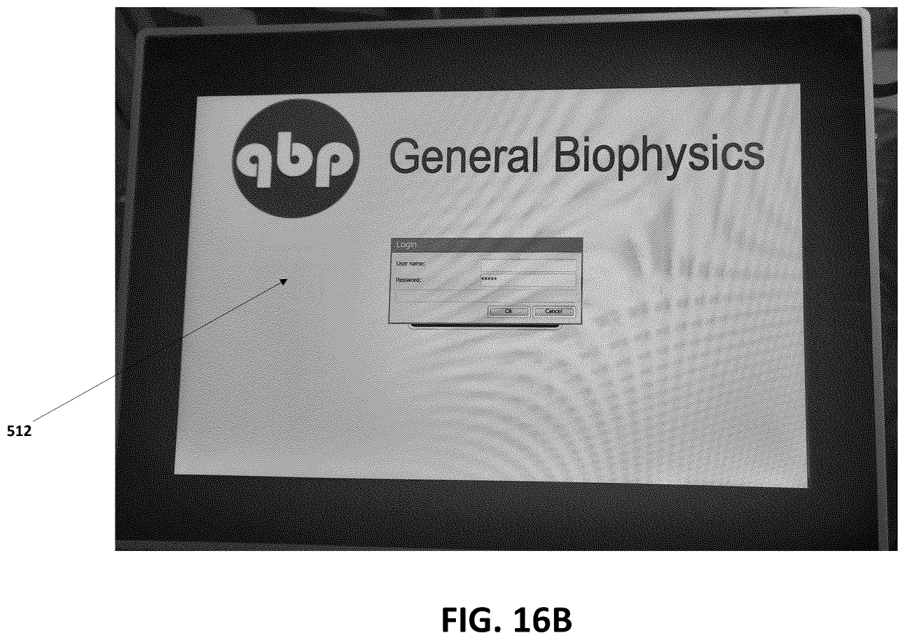

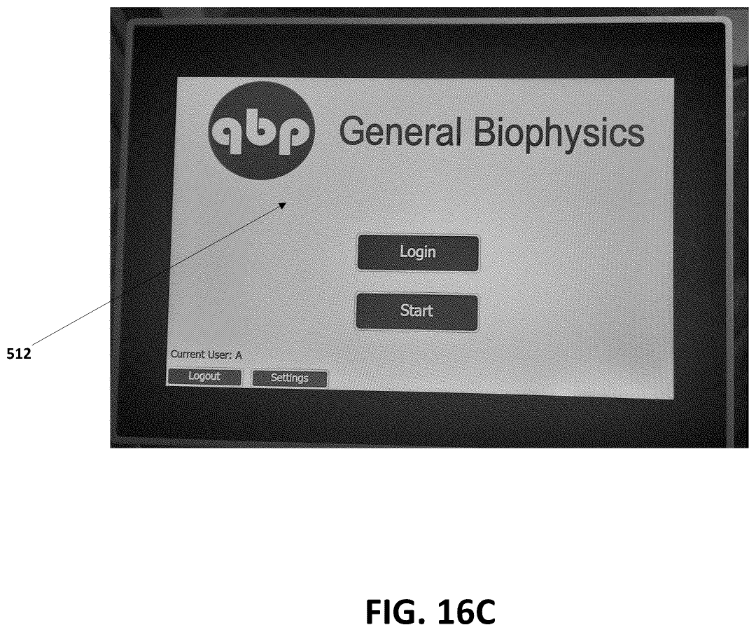

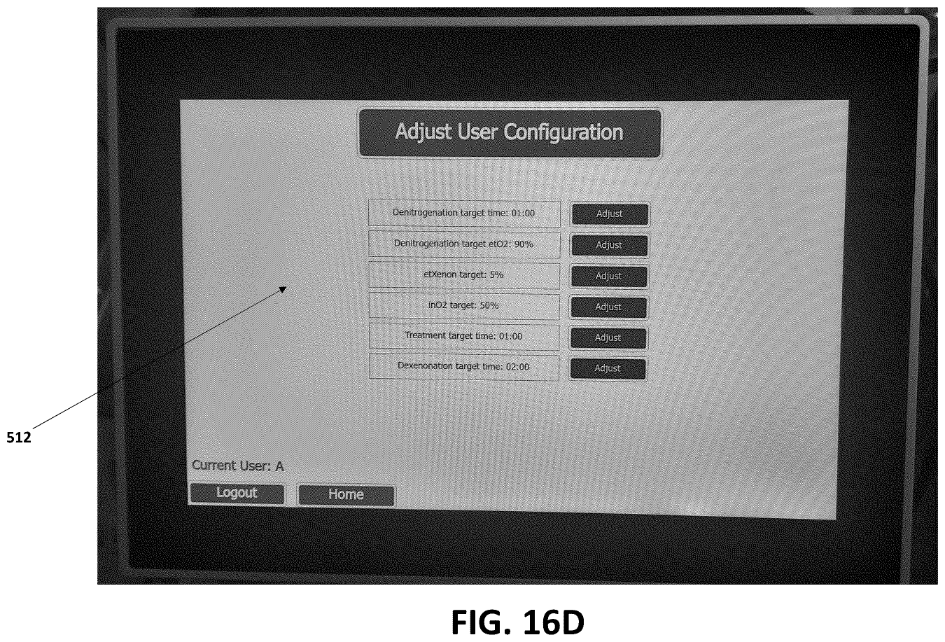

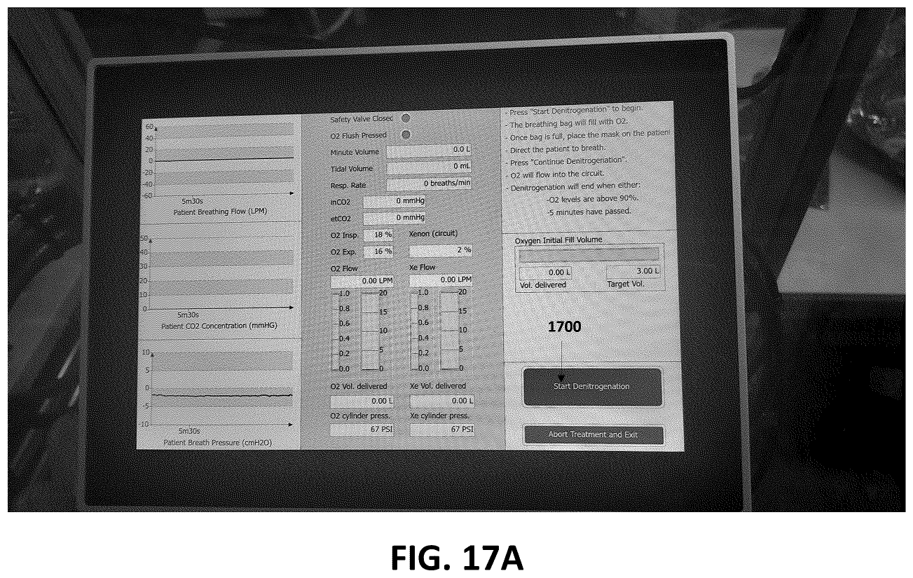

FIGS. 16A-16D illustrate a password-protected management system 1600 displayed on the touchscreen 108. Upon powering on the inhalation system 100, the user is required to log in to the password-protected management system 1600, as shown in FIGS. 16A-16B. Furthermore, a user may be either an operator or an administrator. An operator can log in to use the system but is unable to adjust any of the device settings. Conversely, an administrator can log in to both use the system and change the device settings, as shown in FIGS. 16C-16D. This prevents accidental deployment of the treatment and deployment of the treatment with the wrong settings, while still allowing for the setting adjustment if necessary.

FIGS. 17A-17E illustrate the user interface (UI) 512 of the inhalation system 100 as displayed on the touchscreen 108 during the treatment of a patient. The treatment can be divided into four main phases, with the UI 512 changing slightly during each phase. Prior to the treatment, as shown in FIG. 17A, the UI 512 displays a button 1700 labeled “Start Denitrogenation.” Upon pressing the button 1700, the treatment enters the first phase in which the patient breathing bag 132 fills with about 3 liters of O₂. As shown in FIG. 17B, the UI 512 shows the volume of O₂ delivered to the breathing circuit as a progress bar 1702. After the patient breathing bag 132 fills, the label of the button 1700 changes to “Continue Denitrogenation.”

Upon pressing the button 1700 now labeled “Continue Denitrogenation,” the treatment enters the second phase, and the UI 512 displays the etO₂ concentration (expiratory O₂ percentage) 1704, and a timer progress bar 1706, as shown in FIG. 17C. The second phase will end once the timer ends, or the etO₂ concentration 1704 is above a certain threshold. The timer length and O₂ threshold can be set by an administrator.

Once the second phase ends, the system automatically proceeds to the third phase, the xenon treatment. Xenon gas is dispensed into the breathing loop 104, along with a background flow of O₂. The flow of each gas is adjusted in order to have 20% Xe in the breathing loop 104, while maintaining high levels of O₂. The xenon concentration and treatment timer are displayed as progress bars 1708, 1710 on the UI 512, as shown in FIG. 17D. The xenon concentration and treatment length can be set by an administrator.

Once the third phase ends, the system automatically proceeds to the fourth phase, dexenonation. During this phase, a high flow of O₂ is dispensed into the breathing loop 104 for a set time. The xenon concentration 1708 and dexenonation timer 1712 are displayed on the UI 512, as shown in FIG. 17E. The target time can be set by an administrator. In some embodiments, during this phase, the exhaled gas is directed into the recovery system 932.

The UI 512 necessary to begin treatment is only accessible to administrators and operators that log into the password-protected management system 1600. Moreover, as mentioned, if the user is an operator, they are able to begin a treatment using the UI 512, but they are unable to change any of the preset settings. The treatment occurs automatically according to the settings previously set by an administrator. Because of this feature, in conjunction with the constant monitoring by the system sensors, the system can be used to provide therapeutic xenon treatments without the presence of an anesthesiologist.

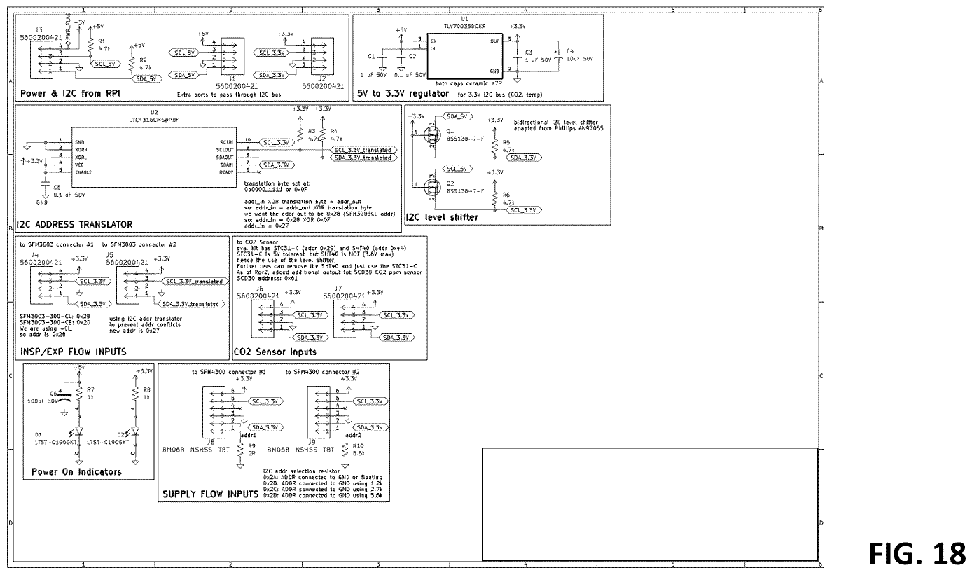

FIG. 18 is a circuit diagram of the I2C PCB 1200. The I2C PCB 1200 preferably comprises an RPI 1800 further comprising the I2C bus 110. The I2C bus 110 allows the RPI 1800 to connect to the flow sensors 508, 522 of the medium pressure system 202, the inspiratory flow sensor 904 of the breathing loop 104, the expiratory flow sensor 918 of the breathing loop 104, the CO₂ sensor 906 of the breathing loop 104, and the CO₂ percentage sensor 916 of the breathing loop 104.

In one preferred embodiment, the I2C PCB 1200 further comprises an I2C address translator 1802, allowing multiple devices to co-exist on the I2C bus 110. The connection of multiple devices to the I2C bus 110 further requires that the I2C PCB 1200 preferably comprise a I2C level shifter 1804 to allow the devices with different voltage requirements to communicate effectively. Moreover, the I2C PCB 110 also preferably comprises a 5V to 3.5V regulator 1806.

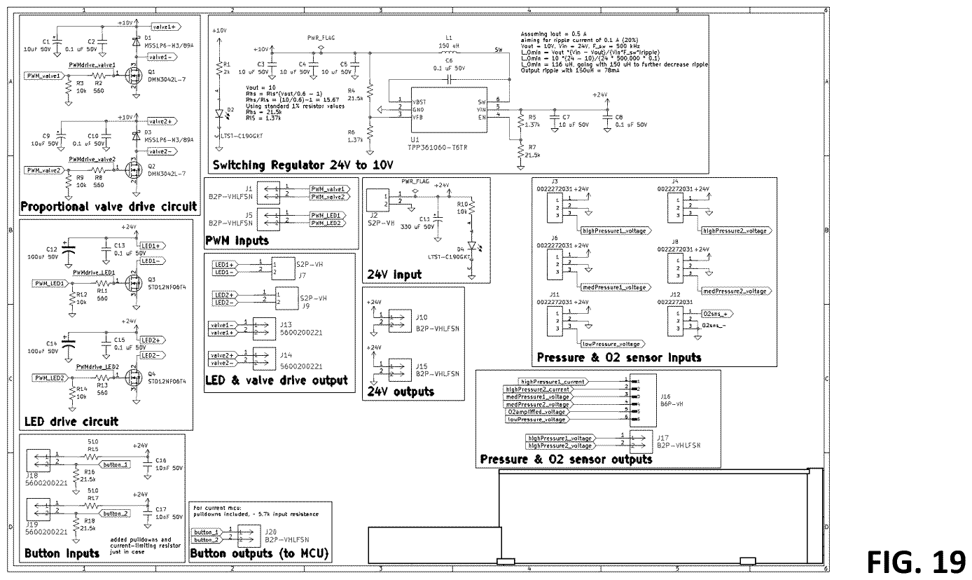

FIG. 19 is a circuit diagram of a controller 106 of the inhalation system 100. The controller 106 further comprises a set of drive circuits 1900, which includes a proportional valve drive circuit 1902 and an LED drive circuit 1904, that provide electrical signals to their respective loads via an LED and valve drive output 1906.

The controller 106 also preferably comprises the set of analog inputs 112 and a set of analog outputs 1908. The sets 112, 1908 are connected to the inspiratory O₂ percentage sensor 910, the expiratory O₂ percentage sensor 920, the xenon pressure sensor 520 of the medium pressure system 202, the oxygen pressure sensor 508 of the medium pressure system 202, the digital pressure sensor 308 of the oxygen high pressure system 204, the digital pressure sensor 404 of the xenon high pressure system 206, and the low pressure sensor 914 of the breathing loop 104.

It should be understood that various alternatives to the embodiments of the disclosure described herein may be employed in practicing the disclosure. Elements of an implementation of the apparatus described herein may be independently implemented or combined with other implementations.

What is claimed is:

1. An automatic inhalation system comprising:
a gas supply system, wherein the gas supply system further comprises at least one high pressure system and at least one medium pressure system;
a breathing loop, wherein a quantity of gas is disposed to enter the breathing loop from the gas supply system; and wherein the breathing loop further comprises an inhale limb and an exhale limb each comprising at least one flow sensor, at least one CO₂ sensor, and at least one O₂ sensor;
a controller, wherein the gas supply system and breathing loop are in digital communication with the controller via an I2C bus and a set of analog inputs by which the gas supply system and the breathing loop provide a set of diagnostic information to the controller; and
a touchscreen disposed to display the set of diagnostic information on a UI accessed through a password-protected user management system, wherein the automatic inhalation system is used to administer gas treatments to patients based on a preset protocol that may be started by either an operator or administrator and edited only by an administrator.

2. The automatic inhalation system of claim 1, wherein the at least one O₂ sensor comprises at least two O₂ sensors.

3. The automatic inhalation system of claim 1, wherein the two O₂ sensors are disposed to calculate the rate of O₂ consumption of the patient and use this information to adjust the background O₂ flow accordingly.

4. The automatic inhalation system of claim 1, wherein the O₂ sensors are disposed to minimize the amount of O₂ used and minimize the amount of xenon needed to be added to the circuit to maintain a stable concentration.

5. The automatic inhalation system of claim 1, wherein the O₂ sensors arrangement provides a safety feature wherein when the patient has a higher-than-normal rate of oxygen consumption, the background flowrate will increase to match the sensed breathing needs of patient.

6. The automatic inhalation system of claim 1, wherein the gas supply system and controller are contained within a housing comprising a front, a back, a left side, a right side and an upper portion comprising a gradient disposed for securely receiving the touchscreen.

7. The automatic inhalation system of claim 1, wherein the high-pressure system further comprises:
an oxygen high pressure system that steps down the pressure of oxygen gas from oxygen cylinder supplies using an oxygen pressure regulator and feeds the oxygen gas into the medium pressure system; and
and a xenon high pressure system that steps down the pressure of xenon gas from a xenon cylinder supply using a xenon pressure regulator and feeds the xenon into the medium pressure system.

8. The automatic inhalation system of claim 1, wherein the medium pressure system further comprises:
an oxygen supply line that receives oxygen gas from the oxygen high pressure system via an electrically controlled proportional solenoid valve, which works only at a designated pressure; and
a xenon supply line that receives xenon gas from the xenon high pressure system via an electrically controlled proportional solenoid valve, which works only at the designated pressure.

9. The automatic inhalation system of claim 8 wherein the designated pressure is in a range from 50-60 psi.

10. The automatic inhalation system of claim 1 wherein the breathing loop further comprises:
a first check valve, wherein the oxygen gas from the oxygen supply line is disposed to be fed through the first check valve and into the breathing loop; and,
a second check valve, wherein the xenon gas from the xenon supply line is disposed to be fed through the second check valve and into the breathing loop.

11. The automatic inhalation system of claim 1 wherein the breathing loop wherein the preferred ratio of xenon:oxygen is 2-3:7-8.

12. The automatic inhalation system of claim 1 wherein the inhale limb further comprises an inspiratory O₂ percentage sensor that measures the percentage of O₂ in the gas mixture in the breathing loop; and wherein the at least one CO₂ sensor of the inhale limb measures the amount of CO₂ in the gas mixture on a level of part per million (ppm) ;
wherein the inspiratory O2 percentage sensor and the at least one CO₂ sensor send their collected data to the controller.

13. The automatic inhalation system of claim 1 wherein the exhale limb further comprises an expiratory O₂ percentage sensor that measures the percentage of O₂ in the exhaled gas mixture in the breathing loop; and wherein the at least one CO₂ sensor of the exhale limb measures percentage of CO₂ in the exhaled gas mixture;
wherein the expiratory O₂ percentage sensor and the at least one CO₂ sensor send their collected data to the controller.

14. The automatic inhalation system of claim 1 wherein the breathing loop further comprises an automatic normally-open safety valve that allows the gas treatment to be interrupted by introducing atmospheric air into the breathing loop in the case of a power outage or gas supply error.

15. The automatic inhalation system of claim 1, further comprising an oxygen flush which allows a user to flood the system with oxygen to prevent hypoxia.

16. The automatic inhalation system of claim 1, wherein the system operates automatically according to the settings previously set by an administrator and wherein the system sensors constantly monitor to provide a set therapeutic xenon treatments without the presence of an anesthesiologist.

17. An automatic inhalation system comprising:
a gas supply system, wherein the gas supply system further comprises at least one high pressure system and at least one medium pressure system;
a breathing loop, wherein a quantity of gas is disposed to enter the breathing loop from the gas supply system; and wherein the breathing loop further comprises an inhale limb and an exhale limb each comprising at least one flow sensor, at least one CO₂ sensor, and at least one O₂ sensor;
a controller, wherein the gas supply system and breathing loop are in digital communication with the controller via an I2C bus and a set of analog inputs by which the gas supply system and the breathing loop provide a set of diagnostic information to the controller; and
a touchscreen disposed to display the set of diagnostic information on a UI accessed through a password protected user management system, wherein the automatic inhalation system is used to administer gas treatments to patients based on a preset protocol that may be started by either an operator or administrator and edited only by an administrator; and,
a portable recovery system.

18. The automatic inhalation system of claim 17, wherein the system operates automatically according to the settings previously set by an administrator and wherein the system Sensors constantly monitor to provide a set therapeutic xenon treatments without the presence of an anesthesiologist.
